(19) United States
(12) Patent Application Publication
(10) Pub. No.: US 2012/0122158 A1
(43) Pub. Date: May 17, 2012

(54) PCR FOR DNA THAT IS RESISTANT TO AMPLIFICATION

(75) Inventors: Steven T. Okino, San Carlos, CA (US); Yanhong Kong, Hercules, CA (US)

(73) Assignee: Bio-Rad Laboratories, Inc., Hercules, CA (US)

(21) Appl. No.: 13/109,688

(22) Filed: May 17, 2011

Related U.S. Application Data
(60) Provisional application No. 61/345,715, filed on May 18, 2010.

(51) Int. Cl. C12P 19/34

(52) U.S. Cl. 435/91.2

(57) ABSTRACT

DNA that is difficult to amplify by conventional PCR is amplified by a modified PCR process that includes a high-temperature, short-term heating step as the last step of each thermal cycle, following the conventional denaturing and annealing/elongation steps.

### CROSS-REFERENCE TO RELATED APPLICATION

This application claims the benefit of U.S. Provisional Patent Application No. 61/345,715, filed May 18, 2010, the contents of which are incorporated herein by reference.

### BACKGROUND OF THE INVENTION

The polymerase chain reaction (PCR) for amplifying DNA, i.e., producing a multitude of copies of DNA from a very small quantity of DNA, is widely used by medical and biological research laboratories in a variety of procedures, including DNA sequencing, functional analyses of genes, diagnoses of hereditary diseases, DNA fingerprinting and forensics, and the detection and diagnosis of infectious diseases.

The typical PCR protocol currently in use is performed in a reaction mixture that includes, in addition to the target or template DNA, primers complementary to the 3′ ends of each of the complementary strands of the target, a DNA polymerase, and deoxynucleoside triphosphates (dNTPs) for each of the four bases that are present in any DNA. Further components of the typical reaction mixture are a buffer and cations such as potassium and either magnesium or manganese to serve as co-factors for the polymerase. As the DNA polymerase, both natural DNA polymerase and laboratory-modified DNA polymerases known as “hot-start” DNA polymerases, which are designed for PCR procedures in which polymerase activity at temperatures below 70° C. is sought to be minimized or eliminated, have been used. When using hot-start DNA polymerase, the protocol begins with a preliminary heating of the reaction mixture at approximately 95° C. or higher, and often from about 95° C. to about 98° C., for about one to about ten minutes, or in many cases about five minutes, to activate the DNA polymerase and to denature the DNA. When using natural DNA polymerase, no such preliminary heating is performed. In either case, the PCR protocol itself entails a series of temperature changes which are repeated in cycles, each cycle including:
(a) Heating at about 95° C. or above, or from about 96° C. to about 98° C. for thirty seconds or less, or about ten seconds to about thirty seconds, and in many cases about fifteen seconds, to achieve denaturation of the target DNA into single-strand DNA, or to complete the denaturation in cases where the preliminary heating step was performed, since such denaturation begins in that preliminary heating; and
(b) An annealing and elongation step performed by reducing the temperature of the reaction mixture to a temperature within the range of about 60° C. to about 72° C., for a period of about one minute, or in some cases, approximately a minute for each thousand base pairs in the original target DNA, to anneal the primers to the single-strand DNA and to elongate the primers once annealed and thereby form DNA strands complementary to (and hybridized to) the single-strand DNA resulting from step (a).

The “cycle” of steps (a) and (b) is typically performed at least twenty times, and often from twenty to fifty times (a common number of cycles presently used is thirty-nine). In the last cycle, step (b) is in some cases continued for a total of about five minutes to about fifteen minutes.

The protocol described above does not serve equally well for all DNA templates. Certain DNA templates require unusually long reaction times to complete the annealing and elongation in step (b), or exhibit low efficiency in this step, or both. Among these amplification-resistant templates are those with relatively long DNA regions, i.e., 250 or more base pairs, such as genomic DNA, and those that are GC-rich, i.e., where the GC base pairs constitute 60% or more of the total base pairs. The present invention provides means for raising the efficiency of PCR for these templates, as well as PCR for DNA templates in general.

### DESCRIPTION OF THE INVENTION

The present invention resides in the discovery that PCR is generally made more efficient by the inclusion of an additional step in the cycle. The additional step is an elongation step performed at high temperature for a short duration, performed at the end of each cycle, i.e., after step (b) above, and before returning to step (a) to begin the next cycle. The temperature at which this additional step is performed is about 75° C. or higher, or from about 75° C. to about 90° C., or in many cases about 78° C. or higher, or from about 78° C. to about 90° C., and the duration is about one minute or less, or about fifteen seconds to about one minute. In many cases, heating at this temperature for about thirty seconds will provide excellent results.

As noted above, the present invention is particularly useful in the amplification by PCR of DNA targets with an amplicon size of 250 or more base pairs, or from 250 base pairs to 3,000 base pairs, and DNA targets that are rich in GC base pairs, including GC contents of 60% or greater, and in many cases those having from about 60% to about 75% GC contents. Examples of genes from the human genome that are expected to exhibit improved amplification efficiency by the practice of the present invention are DKK1, TP73, and CDH1.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. Any discrepancy between any teaching in a published document or in any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A process for amplification of a target DNA by polymerase chain reaction, said process comprising multiple repeats of a cycle that comprises:
(a) with said target DNA dissolved in a PCR reaction mixture comprising said target DNA, primers, a DNA polymerase, and deoxynucleoside triphosphates, heating said PCR reaction mixture sufficiently to achieve denaturation of said target DNA into single-strand DNA;
(b) cooling said PCR reaction mixture sufficiently to cause said primers to anneal to said single-strand DNA and to elongate and thereby at least partially form DNA strands complementary to said single-strand DNA; and
(c) subjecting said PCR reaction mixture to a temperature of about 75° C. or higher for about one minute or less to further elongate said complementary DNA strands formed in step (b).

2. The process of claim 1 wherein said target DNA comprises a minimum of 250 base pairs.

3. The process of claim 1 wherein said target DNA comprises from 250 base pairs to 3,000 base pairs.

4. The process of claim 1 wherein said target DNA has as GC content of about 60% or greater.

5. The process of claim 1 wherein said target DNA has as GC content of from about 60% to about 75%.

6. The process of claim 1 wherein said temperature of step (c) is within the range of about 75° C. to about 90° C.

7. The process of claim 1 wherein said temperature of step (c) is within the range of about 78° C. to about 90° C.

8. The process of claim 1 wherein step (c) is performed at a temperature within the range of about 75° C. to about 90° C. and for a period of from about fifteen seconds to about one minute.

9. The process of claim 1 wherein step (c) is performed at a temperature within the range of about 78° C. to about 90° C. and for a period of from about fifteen seconds to about one minute.

10. The process of claim 1 wherein step (a) is performed at a temperature of about 95° C. or higher.

11. The process of claim 1 wherein step (a) is performed at a temperature of from about 96° C. to about 98° C. for a period of from about ten seconds to about thirty seconds.

12. The process of claim 1 further comprising:
an initial heating step prior to said multiple repeats, said initial heating step comprising heating said PCR reaction mixture to a temperature of about 95° C. or higher, for a period of from about one minute to about nine minutes, and
a final cycle comprising a single performance of step (a) followed by a single performance of step (b) for a period of from about five minutes to about fifteen minutes.

13. The process of claim 1 wherein step (b) is performed at a temperature of from about 60° C. to about 72° C. for a period of about from about thirty seconds to about two minutes for each thousand base pairs in said target DNA.
